(19) United States
(12) Patent Application Publication
(10) Pub. No.: US 2004/0024362 A1
(43) Pub. Date: Feb. 5, 2004

(54) Stabilizing surgical delivery apparatus and method of use

(76) Inventors: Hugh Trout III, Bethesda, MD (US); Frank Patterson, Exeter, NH (US)

Correspondence Address: COLLIER SHANNON SCOTT, PLLC, 3050 K STREET, NW, SUITE 400, WASHINGTON, DC, 20007, US

(21) Appl. No.: 10/410,924

(22) Filed: Apr. 11, 2003

Related U.S. Application Data
(60) Provisional application No. 60/371,432, filed on Apr. 11, 2002.

(51) Int. Cl. A61M 5/32

(52) U.S. Cl. 604/174

(57) ABSTRACT

An embodiment of the present invention relates to an apparatus and method for stabilizing a catheter during a surgical procedure. In particular, an embodiment of the present invention relates to a stabilizing surgical delivery apparatus that comprises a catheter and a plurality of micro-barbs located on an external surface of the catheter, wherein the micro-barbs are minimally exposed when the catheter is in a linear position and fully exposed when the catheter is in an articulated, non-linear position. The micro-barbs contact or may become embedded in an endolumenal surface opposite the surgical site to provide stabilization to the catheter during the surgical procedure.

### CROSS-REFERENCE TO RELATED APPLICATIONS

The present invention relates to, and is entitled to the benefit of the earlier filing date and priority of, U.S. Provisional Patent Application No. 60/371,432, filed Apr. 11, 2002.

### FIELD OF THE INVENTION

An embodiment of the present invention relates to an apparatus and method for stabilizing a catheter during a surgical procedure. In particular, an embodiment of the present invention relates to a catheter having a plurality of micro-barbs for contacting a surface to discourage dislocation and slippage of the catheter during a surgical procedure.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because no statistical benefit exists in performing such procedures.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is high, there is also considerable mortality and morbidity associated with open surgical intervention to repair an aortic aneurysm. This intervention involves penetrating the abdominal wall to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same

Although in recent years certain techniques have been developed that may reduce the stress, morbidity, and risk of mortality associated with surgical intervention to repair aortic aneurysms, including delivery catheter assemblies, none of the systems that have been developed provide an apparatus that assists in stabilization of a catheter during a surgical procedure by contact between a surface, such as a surgical component such as a graft, or a vessel wall, and a micro-barbed or otherwise textured surface. An embodiment of the present invention provides an apparatus and method for assisting in maintaining the angle and apposition of the catheter to discourage dislocation and slippage of the catheter during the placement of one or more surgical fasteners or during any other surgical procedure. In particular, an embodiment of the apparatus utilizes a plurality of micro-barbs disposed on a catheter, which are embedded in a surface opposite from the surgical site. In an embodiment, the micro-barbs discourage movement of the catheter as an inner sheath of the catheter is extended and during placement of one or more surgical fasteners. In an alternative embodiment the apparatus utilizes a textured surface. An embodiment of the apparatus assists in positional accuracy and placement depth of surgical fasteners at the surgical site. Further, the apparatus of an embodiment of the present invention provides stabilization from within the vessel, reducing the intrusiveness of the surgical procedure. In an embodiment of the present invention the exposure of the micro-barbs may be adjusted as desired.

It is therefore an advantage of an embodiment of the present invention to provide an apparatus and method for facilitating the repair of aortic aneurysms. It is an advantage of an embodiment of the present invention to provide an apparatus for assisting in the stabilization of a catheter during a surgical procedure. It is another advantage of an embodiment of the present invention to provide an apparatus for resisting forces imposed on a catheter during extension of an inner catheter sheath and placement of surgical fasteners. It is an advantage of an embodiment of the present invention to provide an apparatus for assisting in maintaining the angle and apposition of the tip of a catheter with respect to a surface. It is another advantage of an embodiment of the present invention to provide an apparatus for discouraging slippage of a catheter on a surface. It is a further advantage of an embodiment of the present invention to provide an apparatus for assisting in maintaining the positioning of the catheter from within a vessel. It is an advantage of an embodiment of the present invention to provide an apparatus for assisting in achieving positional accuracy and desired placement depth of surgical fasteners.

Additional advantages of embodiments of the present invention are set forth, in part, in the description which follows and, in part, will be apparent to one of ordinary skill in the art from the description and/or from the practice of embodiments of the invention.

### SUMMARY OF THE INVENTION

Responsive to the foregoing challenges, Applicant has developed an innovative stabilizing surgical delivery apparatus, comprising a catheter and a plurality of micro-barbs located on an external surface of the catheter, wherein the micro-barbs are minimally exposed when the catheter is in a linear position and fully exposed when the catheter is in an articulated, non-linear position. The micro-barba may also be comprised of a textured surface.

In another embodiment of the present invention the stabilizing surgical delivery apparatus comprises a catheter and a removable component positioned about a distal end portion of the catheter, the component having an outer surface with a plurality of micro-barbs located thereon, wherein the micro-barbs are minimally exposed when the catheter is in a linear position and fully exposed when the catheter is in an articulated, non-linear position. The removable component may comprise a sleeve, or alternatively, a strip.

Another embodiment of the stabilizing surgical delivery apparatus comprises a catheter, a plurality of micro-barbs located on an external surface of a distal end portion of the catheter, and a control wire with a first end and a second end, wherein a second end extends from a point of proximal access to the micro-barbs to which the first end is attached for adjusting the extension of the micro-barbs.

In accordance with another embodiment of the present invention, the stabilizing surgical delivery apparatus comprises a catheter, a plurality of micro-barbs located on an external surface of a distal end portion of the catheter, and a sleeve slidably positioned about the catheter for adjusting the exposure of the micro-barbs.

An embodiment of the present invention is also directed to a method for stabilizing a catheter during a surgical procedure, comprising the steps of inserting a catheter having an inner sheath and an outer sheath into a vessel from a point of proximal access, positioning a distal end of the catheter adjacent to a surgical site, articulating a tip at the distal end of the catheter, extending the inner sheath from within the outer sheath of the catheter, and activating a surgical stabilizer of the catheter.

The step of activating a surgical stabilizer may comprise contacting and possibly embedding a plurality of micro-barbs, which are located on an external surface of the catheter and exposed by the articulation of the tip of the catheter, in a surface opposite the surgical site. The surface may comprise a vessel wall or a synthetic surface.

Alternatively, the step of activating a surgical stabilizer may comprise contacting and possibly embedding a plurality of micro-barbs, which are located on a removable component and exposed by the articulation of the catheter tip, in a surface opposite the surgical site. The surface may comprise a vessel wall or a synthetic surface.

In accordance with another embodiment of the present invention, the step of activating a surgical stabilizer may comprise pulling a control wire attached to a plurality of micro-barbs, which are located on an external surface of the catheter, thereby extending the barbs and contacting and possibly embedding the barbs in a surface opposite the surgical site. The surface may comprise a vessel wall or a synthetic surface.

The step of activating a surgical stabilizer may alternatively comprise retracting a sleeve that is slidably positioned about the catheter, thereby exposing a plurality of micro-barbs located on an external surface of the catheter and contacting and possibly embedding the barbs in a surface opposite the surgical site. The surface may comprise a vessel wall or a synthetic surface.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of embodiments of the present invention.

### BRIEF DESCRIPTION OF THE DRAWINGS

In order to assist the understanding of this invention, reference will now be made to the appended drawings, in which like reference numerals refer to like elements. The drawings are exemplary only, and should not be construed as limiting the invention.

FIG. 1a is a perspective view of the stabilizing surgical delivery apparatus of an embodiment of the present invention in contact with a surface.

FIG. 1b is an illustration of the micro-barbs located on the surface of the catheter when the catheter is in a linear position.

FIG. 1c is an illustration of the micro-barbs located on the surface of the catheter when the catheter is in an articulated, non-linear position.

FIG. 1d is a perspective view of a removable component in accordance with an embodiment of the present invention having a micro-barbed surface for positioning about a catheter.

FIG. 2a is a perspective view of the stabilizing surgical delivery apparatus of an embodiment of the present invention having micro-barbs located on the surface of a catheter with a control wire attached thereto.

FIG. 2b is an illustration of the extension of the micro-barbs when the control wire is actuated.

FIG. 3a is an illustration of the stabilizing surgical delivery apparatus of an embodiment of the present invention having a slidable sleeve covering a plurality of micro-barbs located on the surface of a catheter.

FIG. 3b is an illustration of the retraction of the slidable sleeve to expose the micro-barbs located on the surface of the catheter.

FIG. 4a is a perspective view of the insertion of a catheter into a vessel to reach a position adjacent to a surgical site.

FIG. 4b is a perspective view of the articulation of the distal tip of the catheter.

FIG. 4c is a perspective view of the extension of an inner sheath of the catheter, resulting in contact between an outer sheath of the catheter and the endolumenal surface opposite from the surgical site.

FIG. 4d is a perspective view of the activation of the surgical stabilizing delivery apparatus.

### DETAILED DESCRIPTION OF EMBODIMENTS OF THE INVENTION

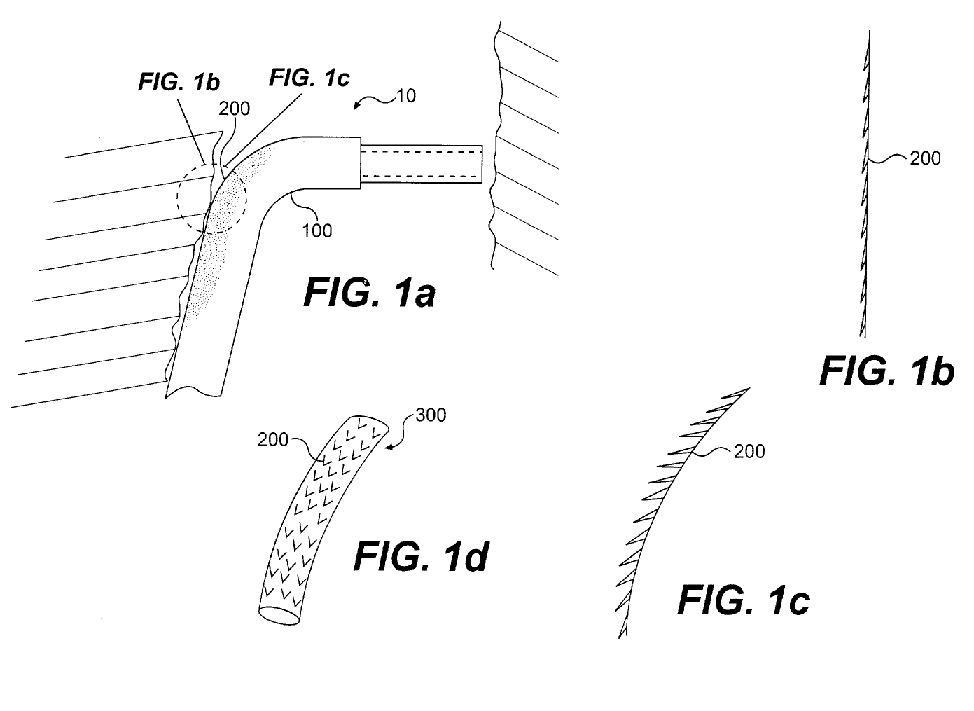

Reference will now be made in detail to an embodiment of the present invention, an example of which is illustrated in the accompanying drawings. With reference to FIGS. 1a through 1c, an embodiment of the stabilizing surgical delivery apparatus 10 comprises a catheter 100 and a plurality of micro-barbs 200 located on an external surface of a distal end portion of the catheter 100. The distal end portion where the barbs 200 are located is the portion that contacts a surface opposite the surgical site during apposition. The micro-barbs 200 are minimally exposed when the catheter 100 is in a linear position, as shown in FIG. 1b. Meanwhile, the micro-barbs 200 are fully exposed when the catheter 100 is in a non-linear position, as shown in FIG. 1c, due to articulation of the tip of the catheter 100. In their fully exposed positioning, the micro-barbs 200 contact, and may become embedded in a surface, such as, but not limited to, a surgical component or a vessel wall, to stabilize the catheter 100.

In accordance with the alternative embodiment depicted in FIG. 1d, the stabilizing surgical delivery apparatus comprises a catheter and a removable component 300 positioned about a distal end portion of the catheter 100. A plurality of micro-barbs 200 reside on the outer surface of the removable component 300. The removable component 300 may comprise a sleeve or a strip or any other suitable device. The component 300 allows the user to remove the micro-barbs 200 if a non-barbed surface is desired for the surgical procedure.

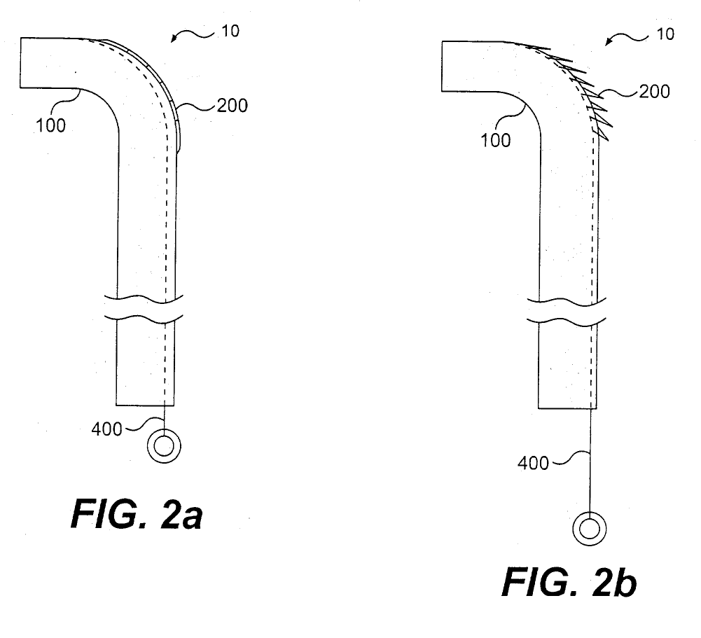

Another embodiment of the present invention is shown in FIGS. 2a and 2b. The stabilizing surgical delivery apparatus 10 comprises a catheter 100, a plurality of micro-barbs 200 located on the external surface of a distal end portion of the catheter 100, and a control wire 400. The control wire 400 extends from a point of proximal access to the patient's body to the micro-barbs 200 located on the catheter 100 and is attached to the micro-barbs 200. The control wire 400 provides for adjustment of the extension of the barbs 200 during a surgical procedure. In accordance with this embodiment of the apparatus, the plurality of micro-barbs 200 may comprise, but is not limited to, about 1 to 10 micro-barbs, but can extend to any suitable number of micro-barbs. In an alternative embodiment, the control wire may adjust a protective cover disposed over the micro-barbs 200.

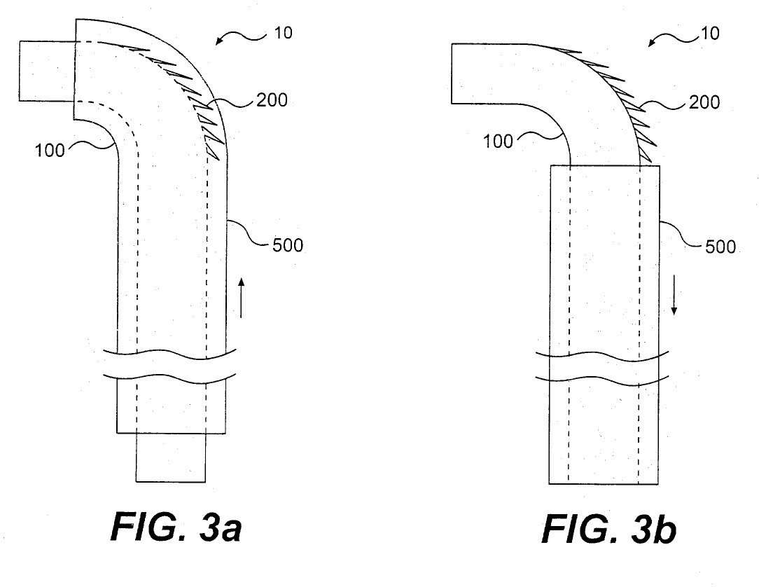

FIGS. 3a and 3b depict another embodiment of the present invention. The stabilizing surgical delivery apparatus 10 comprises a catheter 100, a plurality of micro-barbs 200 located on the external surface of a distal end portion of the catheter 100, and a sleeve 500 slidably positioned about the catheter 100. When stabilization of the catheter 100 is not desired, the sleeve 500 covers the micro-barbs 200, as illustrated in FIG. 3a. The sleeve 500 may be retracted, when desired, to expose the micro-barbs 200, as illustrated in FIG. 3b.

In an embodiment, the micro-barbs 200 may have a diameter in a range of between about 0.005 inches and about 0.010 inches, but may be any suitable diameter such as, but not limited to about 0.0005 inches to about 0.10 inches. The length of the micro-barbs 200 may be in a range of between about 0.010 inches and about 0.100 inches, but may be of any suitable length such as, but not limited to about 0.001 inches to about 0.50 inches. The micro-barbs 200 may comprise a series of metallic wires, or any other suitable material, or alternatively, the barbs 200 may be formed from surface texturing techniques, such as ion texturing or EDM , or any other suitable method of forming the textured surface. The microbarbs may be shaped to clutch the surface such as, but not limited to, a wedged shape where the tip of the wedge comes in contact with the surface or a wire or any appropriate configuration.

In an embodiment, the surface may comprise, but is not limited to, a vessel or tissue wall or a synthetic surface, such as a graft, or any other surgical component. The synthetic surface may comprise, but is not limited to, polyester, either woven or knitted, or expanded PTFE stent-graft or unsupported graft. The contacting, embedding or attachment of the micro-barbs to the surface may be atraumatic and may not be injurious to either the natural or the synthetic surface.

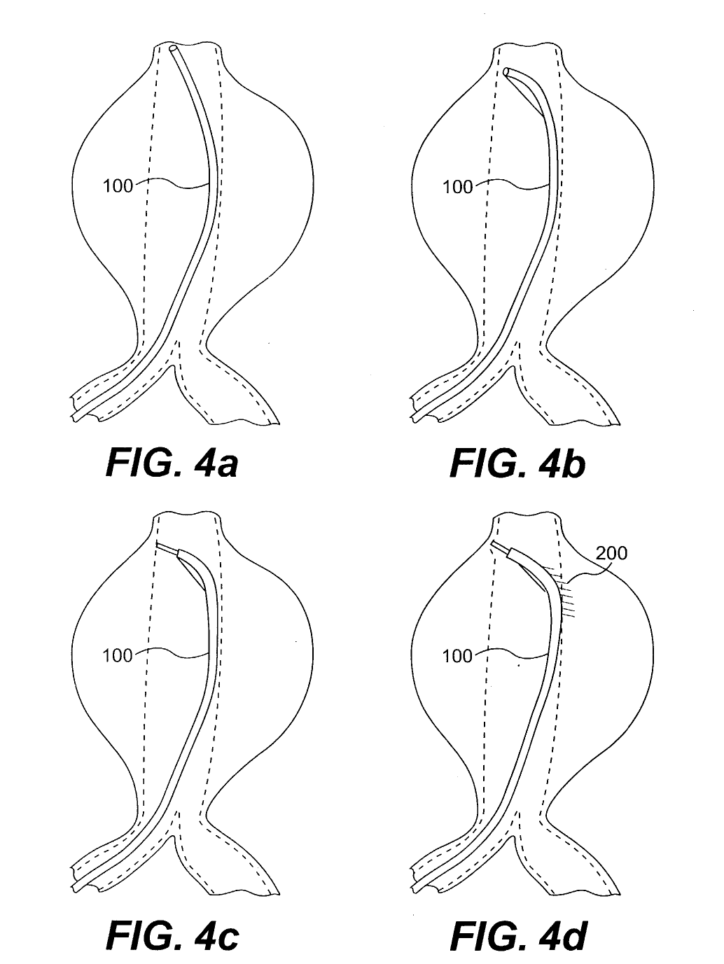

The operation of the stabilizing surgical delivery apparatus will now be described in accordance with FIGS. 4a through 4d. A catheter 100 is inserted into a vessel from a point of proximal access to the patient's body. The distal end of the catheter 100 is positioned adjacent to the surgical site, as shown in FIG. 4a. The distal tip of the catheter 100 is articulated (i.e., adjusted to an angular configuration), as shown in FIG. 4b. The articulation may be created by at least one pull wire positioned within an outer sheath of the catheter 100, as disclosed in U.S. patent application Ser. No. 09/783,313, filed Feb. 15, 2001, which is incorporated herein by reference, or by any other suitable means. The tip is deflected when the pull wire is tensioned. Once the tip has been deflected to the desired angle, an inner sheath of the catheter 100 is extended from within an outer sheath of the catheter 100, as depicted in FIG. 4c. The inner sheath is extended into contact with an endolumenal surface, in particular a repair member, surgical component, or a vessel wall, at the surgical site. Continued extension of the inner sheath forces the outer sheath of the catheter 100 into contact with the endolumenal surface opposite the surgical site (referred to as apposition), as disclosed in U.S. patent application Ser. No. 09/783,313, which is herein incorporated by reference. The surgical stabilizer is then activated to discourage dislocation and slippage of the catheter 100 during the surgical procedure, as shown in FIG. 4d.

The step of activating the surgical stabilizer includes contacting or possibly embedding a plurality of micro-barbs into the surface opposite the surgical site. In accordance with the embodiment of the apparatus 10 depicted in FIGS. 1a through 1c, the micro-barbs 200 located on the external surface of a distal end portion of the catheter 100 are exposed by the articulation of the tip of the catheter 100. After extension of the inner sheath of the catheter 100, resulting in apposition, the micro-barbs 200 contact and may become embedded in the surface opposite from the surgical site to assist in stabilizing the catheter 100. Similarly, in accordance with the embodiment depicted in FIG. 1d, the micro-barbs 200 located on a removable component 300 may be exposed by the articulation of the tip of the catheter 100. Once apposition is achieved, the barbs 200 contact or may become embedded in the endolumenal surface opposite the surgical site.

As shown in FIGS. 2a and 2b the step of activating the surgical stabilizer may include pulling or otherwise actuating a control wire 400, which may include a wire, or any other suitable material, that is attached to a plurality of micro-barbs 200 residing on the external surface of a distal end portion of the catheter 100. The barbs 200 are extended when the wire 400 is pulled, as illustrated in FIG. 2b, and the barbs 200 then contact or may become embedded in the endolumenal surface opposite the surgical site. Further, the barbs 200 may be retracted into conformance with the external surface of the catheter 100 by pushing or otherwise actuating the control wire 400 once stabilization of the catheter 100 is no longer desired. The control wire 400 may also be used to activate a protective cover disposed over the micro-barbs 200.

In accordance with the embodiment shown in FIGS. 3a and 3b, the stabilizer is activated by retracting a sleeve 500 that is slidably positioned about the catheter 100. When the sleeve 500 is retracted, a plurality of micro-barbs 200 located on the external surface of a distal end portion of the catheter 100 are exposed, as depicted in FIG. 3b. Once exposed, the micro-barbs 200 contact or may become embedded in the surface opposite the surgical site. When stabilization of the catheter 100 is no longer desired, the sleeve 500 may be repositioned to cover the micro-barbs 200.

It will be apparent to those skilled in the art that variations and modifications of embodiments of the present invention can be made without departing from the scope or spirit of the invention. For example, an embodiment of the present invention is not limited to stabilizing a catheter during attachment of a repair member or surgical component, which may be a graft, to a vessel wall. Rather, it is contemplated that an embodiment of the present invention may be used in connection with securing a vessel to another vessel, tissue to tissue, surgical components to surgical components, and any variations thereof. Thus, it is intended that an embodiment of the present invention cover all such modifications and variations of the invention, provided they come within the scope of the appended claims and their equivalents.

What is claimed is:

1. A surgical stabilizer for use with a catheter comprising:
a plurality of micro-barbs having a first end and a second end, wherein the first end is substantially free to contact a surface and the second end is disposed on an external surface of the catheter.

2. The surgical stabilizer according to claim 1, wherein the micro-barbs are minimally exposed when the catheter is in a linear position and fully exposed when the catheter is in a non-linear position.

3. The surgical stabilizer according to claim 1, further comprising a control wire with a first end and a second end, wherein the first end is in communication with the micro-barbs and the second end extends away from the surgical site.

4. The apparatus according to claim 3, wherein the plurality of micro-barbs comprises from about 1 to 10 micro-barbs.

5. The surgical stabilizer according to claim 1, further comprising a sleeve slidably positioned about the catheter for adjusting the exposure of the micro-barbs.

6. A stabilizing surgical delivery apparatus, comprising:
a catheter; and
a removable component positioned about a distal end portion of the catheter, the component having an outer surface with a plurality of micro-barbs located thereon, wherein the micro-barbs are minimally exposed when the catheter is in a linear position and fully exposed when the catheter is in an articulated, non-linear position.

7. The apparatus according to claim 6, wherein the removable component comprises a sleeve.

8. The apparatus according to claim 6, wherein the removable component comprises a strip.

9. A method for stabilizing a catheter during a surgical procedure, comprising the steps of:
inserting a catheter into a vessel from a point of proximal access;
positioning a distal end of the catheter adjacent to a surgical site;
articulating the distal end of the catheter; and
activating a surgical stabilizer on the catheter.

10. The method according to claim 9, wherein the step of activating a surgical stabilizer comprises contacting a plurality of micro-barbs disposed on an external surface of the catheter with a surface opposite the surgical site.

11. The method according to claim 10, wherein the surface opposite the catheter comprises a vessel wall.

12. The method according to claim 10, wherein the surface opposite the catheter comprises a synthetic surface.

13. The method according to claim 9, wherein the step of activating a surgical stabilizer comprises contacting a plurality of micro-barbs disposed on a removable component to a surface opposite the surgical site.

14. The method according to claim 13, wherein the surface comprises a vessel wall.

15. The method according to claim 13, wherein the surface comprises a synthetic surface.

16. The method according to claim 9, wherein the step of activating a surgical stabilizer comprises articulating a control wire attached to the micro-barbs to extend the barbs.

17. The method according to claim 9, wherein the step of activating a surgical stabilizer comprises displacing a sleeve that is slidably positioned about the catheter wherein exposing a plurality of micro-barbs located on an external surface of the catheter.

18. A method for using a stabilizing surgical delivery apparatus comprising the steps of:
inserting a catheter of the stabilizing surgical delivery apparatus into a vessel from a point of proximal access;
positioning the catheter adjacent to a surgical site;
articulating the catheter; and
activating a plurality of micro barbs of the stabilizing surgical delivery apparatus.

19. The method of claim 18, wherein the step of activating the micro barbs of the stabilizing surgical delivery apparatus comprises contacting the micro barbs disposed on an external surface of the catheter with a surface opposite the surgical site.

20. The method of claim 18, wherein the step of activating a plurality of micro barbs comprises articulating a control wire attached to the micro barbs wherein extending the bards.
